(19) United States
(12) Patent Application Publication
(10) Pub. No.: US 2025/0273310 A1
(43) Pub. Date: Aug. 28, 2025

(54) DETERMINATION DEVICE, DETERMINATION METHOD, AND RECORDING MEDIUM

(71) Applicant: NEC Corporation, Tokyo (JP)

(72) Inventors: Kazunari TAKAYAMA, Tokyo (JP); Akira YAMAUCHI, Tokyo (JP); Mayu KAYA, Tokyo (JP); Yasunari TAKEI, Tokyo (JP); Junya USHIKOSHI, Tokyo (JP); Junichi YAHARA, Tokyo (JP)

(73) Assignee: NEC Corporation, Tokyo (JP)

(21) Appl. No.: 19/003,544

(22) Filed: Dec. 27, 2024

(30) Foreign Application Priority Data
Feb. 28, 2024 (JP) 2024-029012

(51) Int. Cl. G16H 10/60

(52) U.S. Cl. CPC G16H 10/60

(57) ABSTRACT

A determination device includes at least one memory configured to store instructions; and at least one processor configured to execute the instructions to: acquire electronic receipt information about a patient, determine necessity of a detailed description of a symptom of the patient based on the electronic receipt information about the patient using a learned model that has learned electronic receipt information excluding a detailed description of a symptom in a past and a condition requiring a detailed description of a symptom, and output a determination result.

This application is based upon and claims the benefit of priority from Japanese Patent Application No. 2024-029012, filed on Feb. 28, 2024, the disclosure of which is incorporated herein in its entirety by reference.

### TECHNICAL FIELD

The present disclosure relates to a determination device, a determination method, and a recording medium.

### BACKGROUND ART

There is a technique for reducing a burden of creating a document created by a doctor or the like.

For example, Reference Document 1 (JP 2015-138402 A) discloses extracting a written expression described in medical information, interpreting the extracted written expression as a medical fact, extracting a confirmed disease that is a disease diagnosed confirmedly from the medical information, and creating an explanation of a diagnosis basis in the confirmed disease diagnosed confirmedly based on the medical fact.

### SUMMARY

An object of the present disclosure is to provide a determination device or the like that can easily grasp a patient whose detailed description of a symptom is required.

A determination device according to an aspect of the present disclosure includes at least one memory configured to store instructions; and at least one processor configured to execute the instructions to: acquire electronic receipt information about a patient, determine necessity of a detailed description of a symptom of the patient based on the electronic receipt information about the patient using a learned model that has learned electronic receipt information excluding a detailed description of a symptom in a past and a condition requiring a detailed description of a symptom, and output a determination result.

In a determination method according to an aspect of the present disclosure executed by a computer, the method includes acquiring electronic receipt information about a patient, determining necessity of a detailed description of a symptom of the patient based on the electronic receipt information about the patient using a learned model that has learned electronic receipt information excluding a detailed description of a symptom in a past and a condition requiring a detailed description of a symptom, and outputting a determination result.

A recording medium according to an aspect of the present disclosure stores a program for causing a computer to execute acquiring electronic receipt information about a patient, determining necessity of a detailed description of a symptom of the patient based on the electronic receipt information about the patient using a learned model that has learned electronic receipt information excluding a detailed description of a symptom in a past and a condition requiring a detailed description of a symptom, and outputting a determination result.

### BRIEF DESCRIPTION OF THE DRAWINGS

Exemplary features and advantages of the present disclosure will become apparent from the following detailed description when taken with the accompanying drawings in which:

FIG. 1 is a block diagram illustrating a configuration of the determination device according to the present disclosure;

FIG. 2 is a diagram illustrating a hardware configuration in which the determination device according to the present disclosure is implemented by a computer device and its peripheral device;

FIG. 3 is a diagram illustrating an example of output of a determination result according to the present disclosure;

FIG. 4 is a flowchart illustrating an operation of determination according to the present disclosure;

FIG. 5 is a block diagram illustrating a configuration of the determination device according to the present disclosure; and

FIG. 6 is a flowchart illustrating an operation of determination according to the present disclosure.

### EXAMPLE EMBODIMENT

Hereinafter, example embodiments of a determination device, a determination method, a program, and a non-transitory recording medium recording the program according to the present disclosure will be described in detail with reference to the drawings. The present example embodiment does not limit the disclosed technology.

### First Example Embodiment

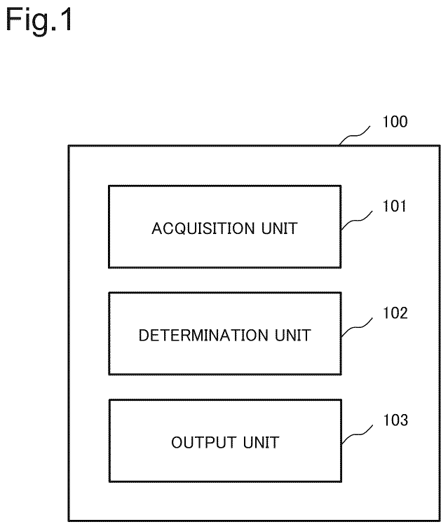

FIG. 1 is a block diagram illustrating an example of a configuration of a determination device 100 according to the present disclosure. As illustrated in FIG. 1, the determination device 100 includes an acquisition unit 101, a determination unit 102, and an output unit 103. The determination device 100 is a device that determines the necessity of “a detailed description of a symptom” that is required to be described in a case where the description of the medical treatment content is considered to be insufficient only with the sick name or the like in the medical fee receipt (receipt). In the detailed description of a symptom, it is necessary to describe the progress of the patient's symptom, the content of the medical act, and the specific reason why the medical act is necessary. The determination device 100 outputs a determination result of necessity of the detailed description of a symptom to a medical professional such as a doctor.

In a case where the detailed description of a symptom is not made for a claim for a medical fee of a medical institution although it is necessary, the examination and payment institution may perform assessment or returning. The assessment means that points are deducted for the content of an item determined to be inappropriate by the examination institution, and the medical fee is paid in the reduced amount. On the other hand, the returning means that the examination institution unilaterally returns the reception itself in a case where it is difficult to determine the appropriateness/inappropriateness of the medical act. When the amount is reduced because the detailed description of a symptom is incomplete, an appropriate consideration for the medical treatment cannot be received. In a case of the returning, it takes time and effort to make a claim again.

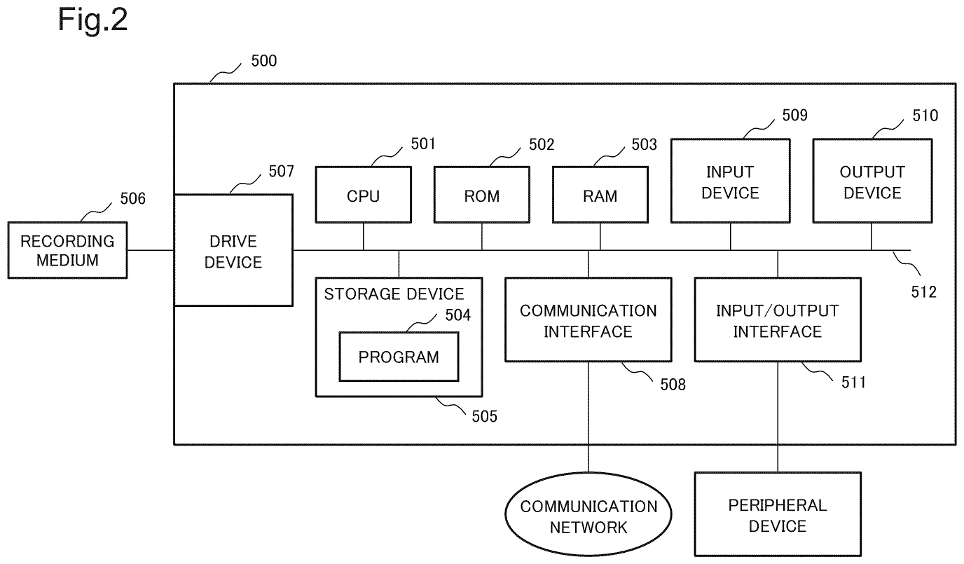

FIG. 2 is a diagram illustrating an example of a hardware configuration in which the determination device 100 in the present disclosure is achieved by a computer device 500 including a processor. As illustrated in FIG. 2, the determination device 100 includes a memory such as a central processing unit (CPU) 501, a read only memory (ROM) 502, and a random access memory (RAM) 503, the storage device 505 such as a hard disk that stores a program 504, a communication interface (I/F) 508 for network connection, and an input/output interface 511 that inputs and outputs data. In the present disclosure, the electronic receipt information acquired by the acquisition unit 101 is input to the determination device 100 via the communication I/F 508.

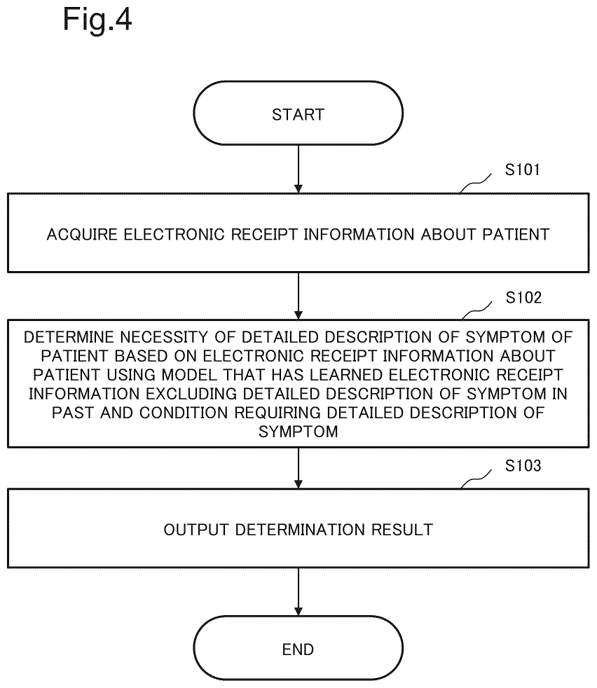

The CPU 501 operates the operating system to control the entire determination device 100 according to the present disclosure. The CPU 501 reads a program and data from a recording medium 506 attached to a drive device 507 or the like to a memory, for example. The CPU 501 functions as the acquisition unit 101, the determination unit 102, the output unit 103, and part thereof according to the present disclosure, and executes processing or a command in the flowchart illustrated in FIG. 4 to be described later based on a program.

The recording medium 506 is, for example, an optical disk, a flexible disk, a magnetic optical disk, an external hard disk, a semiconductor memory, or the like. The recording medium as part of the storage device is a nonvolatile storage device, and records a program therein. The program may be downloaded from an external computer (not illustrated) connected to a communication network.

An input device 509 is achieved by, for example, a mouse, a keyboard, a built-in key button, and the like, and is used for an input operation. The input device 509 is not limited to a mouse, a keyboard, and a built-in key button, and may be, for example, a touch panel. An output device 510 is achieved by, for example, a display, and is used to check an output.

As described above, the determination device 100 illustrated in FIG. 1 is implemented by the computer hardware illustrated in FIG. 2. However, the means for achieving each unit included in the determination device 100 in FIG. 1 is not limited to the above-described configuration. The determination device 100 may be achieved by one physically coupled device, or may be achieved by a plurality of devices by connecting two or more physically separated devices in a wired or wireless manner. For example, the input device 509 and the output device 510 may be connected to the computer device 500 via a network. The determination device 100 illustrated in FIG. 1 can also be configured by cloud computing or the like.

The acquisition unit 101 is a means for acquiring electronic receipt information about a patient. The electronic receipt information is electronic data in which a medical fee receipt is recorded in a text of Comma Separated Values (CSV) format or the like and that a medical institution submits to an examination and payment institution. The acquisition unit 101 acquires electronic receipt information about a patient stored in a server device or the like of a medical institution, to output the electronic receipt information to the determination unit 102.

The acquisition unit 101 may acquire electronic receipt information about a specific patient, or may acquire electronic receipt information about a plurality of patients for each of whom a medical act has been performed in a predetermined period. In a case where the acquisition unit 101 acquires electronic receipt information about a plurality of patients for each of whom a medical act has been performed in a predetermined period, electronic receipt information that requires the detailed description of a symptom and electronic receipt information that does not requires it are mixed. In the electronic receipt information for which the detailed description of a symptom is required, the detailed description of a symptom has not yet been made.

The determination unit 102 is a means for determining the necessity of the detailed description of a symptom of the patient based on the electronic receipt information about the patient using a learned model that has learned the electronic receipt information excluding the detailed description of a symptom in the past and the condition requiring the detailed description of a symptom.

The determination unit 102 inputs electronic receipt information about the patient for determining necessity of the detailed description of a symptom to the learned model to acquire information about a patient who requires the detailed description of a symptom. The learned model is a learned model that has learned receipt information excluding the detailed description of a symptom and a pattern of necessity of the detailed description of a symptom submitted by a medical institution in the past. More specifically, the learned model is generated by machine learning using, as a teacher data set, information obtained by labeling necessity of actual detailed description of a symptom together with receipt information excluding detailed description of a symptom. With respect to electronic receipt information indicated as requiring the detailed description of a symptom of electronic receipt information by returning or assessment, receipt information excluding the detailed description of a symptom and a pattern of necessity of the detailed description of a symptom when submitted by a medical institution after returning or assessment are learned. The receipt information excluding the detailed description of a symptom includes, for example, at least any one of a sick name, content of a medical act, and the score of a claim for a medical fee.

The learned model may be a large-scale language model learned with a large amount of text data or a learned model obtained by transfer learning the large-scale language model. Examples of the large-scale language model can include generative pre-training-2 (GPT-2), GPT-3, or GPT-4. Examples of the large-scale language model may include text-to-text transfer transformer (T5), bidirectional encoder representations from transformers (BERT), robustly optimized BERT approach (RoBERTa), and efficiently learning an encoder that classifies token replacements accurately (ELECTRA). For example, the learned model performs a process according to a prompt input by a medical professional who is a user to output necessity of the detailed description of a symptom. The learned model may be an interactive model that receives and responds to a prompt. The learned model may be stored in the storage device 505 or may be a learned model constructed in an external system.

The prompt includes electronic receipt information for determining necessity of the detailed description of a symptom and an instruction to make the model answer necessity of the detailed description of a symptom. The prompt may include an output condition of a patient for whom it is determined that the detailed description of a symptom is required. The output condition includes, for example, outputting information about a patient related to a specific condition, or outputting a basis of electronic receipt information determined to require the detailed description of a symptom. The specific condition includes, for example, a patient who has visited a specific diagnosis and treatment department or a patient of a specific doctor in charge. The basis of the electronic receipt information is, for example, a case where the score of a claim for a medical fee (high expense receipt) is higher than a predetermined value, a medical act is an act for which the detailed description of a symptom is essential when a medical fee is claimed, or the description of the medical treatment content is insufficient only with a sick name.

The output unit 103 is a means for outputting the determination result on a display device such as a display. The output unit 103 output information about a patient whose detailed description of a symptom is required on, for example, a screen of an application program for browsing electronic receipt information. The output unit 103 may output information about the patient whose detailed description of a symptom is required when the doctor performs an operation of creating the detailed description of a symptom of the patient as a trigger.

In a case where the prompt includes an output condition of a patient for whom it is determined that the detailed description of a symptom is required, the output unit 103 outputs information about the patient related to the condition. For example, in a case where the prompt includes a condition for outputting patients who have visited a specific diagnosis and treatment department, the output unit 103 outputs a list of patients who have visited the diagnosis and treatment department. For example, in a case where the prompt includes a condition for outputting the basis of the electronic receipt information determined to require the detailed description of a symptom, the output unit 103 outputs the basis of the electronic receipt information determined to require the detailed description of a symptom.

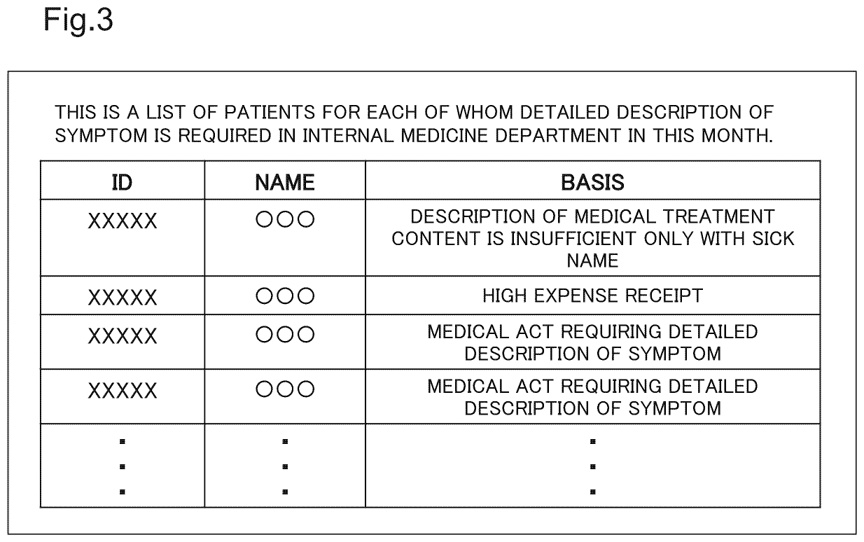

FIG. 3 is an example of output of a determination result according to the present disclosure. The example of FIG. 3 is a list of patients for each of whom it is determined that the detailed description of a symptom is required among patients who have visited internal medicine department of a medical institution. As illustrated in FIG. 3, the output unit 103 may output a basis of electronic receipt information determined to require the detailed description of a symptom, in addition to the ID of the patient and the name of the patient. In the example of FIG. 3, a list of patients for each of whom it is determined that the detailed description of a symptom is required in the internal medicine department is output, but information about patients related to other conditions may be output. For example, the output unit 103 may output a list of patients of a specific doctor in charge.

The operation of the determination device 100 configured as described above will be described with reference to the flowchart of FIG. 4.

FIG. 4 is a flowchart illustrating an outline of the operation of the determination device 100 according to the present disclosure. The processing according to this flowchart may be executed based on program control by the processor described above. The processing according to this flowchart is started, for example, when an operation for displaying information about a patient whose detailed description of a symptom is required is performed as a trigger.

As illustrated in FIG. 4, first, the acquisition unit 101 acquires electronic receipt information about a patient (step S101). Next, the determination unit 102 determines the necessity of the detailed description of a symptom of the patient based on the electronic receipt information about the patient using the learned model that has learned the electronic receipt information excluding the detailed description of a symptom in the past and the condition requiring the detailed description of a symptom (step S102). Finally, the output unit 103 outputs the determination result (step S103). Thus, the determination device 100 ends the determination operation.

In the determination device 100, the determination unit 102 determines the necessity of the detailed description of a symptom of the patient based on the electronic receipt information about the patient using the learned model that has learned the electronic receipt information excluding the detailed description of a symptom in the past and the condition requiring the detailed description of a symptom, and the output unit 103 outputs the determination result. As a result, for example, a doctor who makes the detailed description of a symptom in the electronic receipt information can easily grasp a patient whose detailed description of a symptom is required.

### Modifications

Next, a modification of the present disclosure will be described in detail with reference to the drawings. Hereinafter, description of content overlapping with the above description will be omitted to the extent that the description of the present example embodiment is not unclear. As in the computer device illustrated in FIG. 2, each component in the present modification can be achieved not only by hardware but also by a computer device or software based on program control.

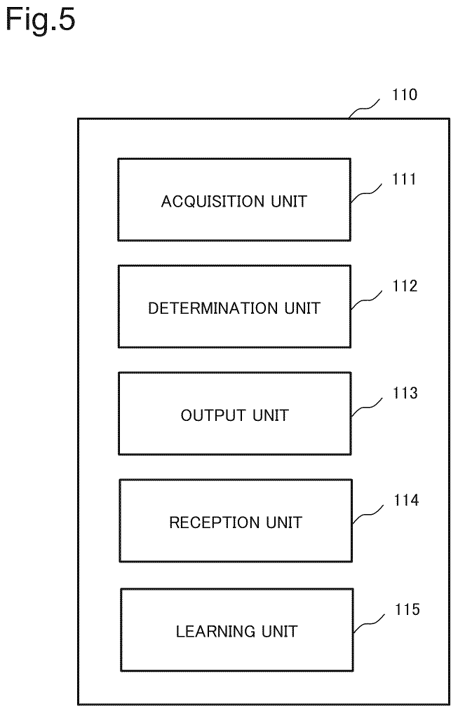

FIG. 5 is a block diagram illustrating a configuration of a determination device 110 in the present disclosure. With reference to FIG. 5, the determination device 110 will be described focusing on a portion different from the determination device 100. The determination device 110 includes an acquisition unit 111, a determination unit 112, an output unit 113, a reception unit 114, and a learning unit 115. The determination unit 112 and the output unit 113 have the same configurations as those of the first example embodiment.

The acquisition unit 111 acquires electronic receipt information about a patient related to the logged-in user. The acquisition unit 111 acquires, for example, electronic receipt information about a patient who has visited a diagnosis and treatment department to which the logged-in user belongs or a patient whom the user is in charge of. The acquisition unit 111 acquires the electronic receipt information about the patient associated with the user name or the user ID from the server device of the medical institution to output the electronic receipt information to the determination unit 112.

The reception unit 114 is a means for receiving a correction of the output determination result. For example, the reception unit 114 receives whether the determination result is correct from the user on the screen that outputs the determination result as to whether the detailed description of a symptom is required. More specifically, the reception unit 114 receives a correction information that the detailed description of a symptom is not required for at least any one of the determination results of the patients for each of whom it is determined that the detailed description of a symptom is required. Alternatively, the reception unit 114 receives correction information that the detailed description of a symptom is required for at least any one of the determination results of the patients for each of whom it is determined that the detailed description of a symptom is not required.

The learning unit 115 is a means for causing the learned model to relearn the electronic receipt information whose correction has been received and the necessity of the detailed description of a symptom as learning data. Specifically, the learning unit 115 changes necessity of the detailed description of a symptom whose correction has been received, and causes the learned model to perform relearning. The learning unit 115 may cause the model to perform relearning every time the correction is received from the user, or may cause the model to perform relearning every predetermined period (for example, every few months).

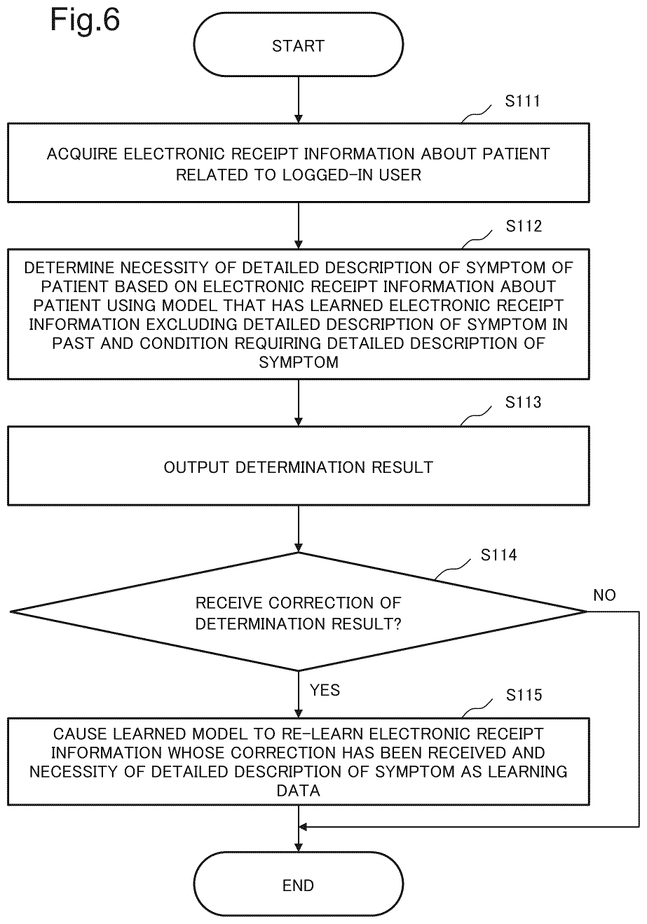

FIG. 6 is a flowchart illustrating an outline of the operation of the determination device 110 in the present disclosure. The processing according to this flowchart may be executed based on program control by the processor described above.

As illustrated in FIG. 6, first, the acquisition unit 111 acquires electronic receipt information about a patient related to the logged-in user (step S111). Next, the determination unit 112 determines the necessity of the detailed description of a symptom of the patient based on the electronic receipt information about the patient using the learned model that has learned the electronic receipt information excluding the detailed description of a symptom in the past and the condition requiring the detailed description of a symptom (step S112). Next, the output unit 113 outputs the determination result (step S113). Next, in a case where the reception unit 114 receives the correction of the output determination result (S114; YES), the learning unit 115 causes the learned model to relearn the electronic receipt information whose correction has been received and the necessity of the detailed description of a symptom as learning data (step S115). On the other hand, when the reception unit 114 does not receive the correction of the output determination result (S114; NO) and ends the flow. Thus, the determination device 110 ends the determination operation.

In the determination device 110 of the present disclosure, the acquisition unit 111 acquires the electronic receipt information about the patient related to the logged-in user, and the determination unit 112 determines the necessity of the detailed description of a symptom of the patient based on the electronic receipt information about the patient using the learned model that has learned the electronic receipt information excluding the detailed description of a symptom in the past and the condition requiring the detailed description of a symptom. The output unit 113 outputs the determination result. In this case, it is possible to confirm only the necessity of the detailed description of a symptom of the patient related to the user.

In the determination device 110 of the present disclosure, in a case where the reception unit 114 receives a correction of the output determination result, the learning unit 115 causes the learned model to relearn the electronic receipt information whose correction has been received and the necessity of the detailed description of a symptom as learning data. As a result, it is possible to output a result reflecting the determination result by the doctor who is the user.

The medical institution creates a medical fee receipt (receipt) at the time of a claim for a medical fee. In a case where it seems that description of the medical treatment content is insufficient only with the sick name, the medical institution is required to describe the details of the medical treatment content in the detailed description of a symptom in the receipt. However, the condition of requiring the detailed description of a symptom is not uniformly determined, and it is difficult to determine in what case the detailed description of a symptom should be made.

According to an example of the effect of the present disclosure, it is possible to easily grasp a patient whose detailed description of a symptom is required.

While the present disclosure is described with reference to example embodiments thereof, the present disclosure is not limited to these example embodiments. Various modifications that can be understood by those of ordinary skill in the art in the art can be made to the configuration and details of the present disclosure within the scope of the present disclosure.

For example, although the plurality of operations is described in order in the form of a flowchart, the order of description does not limit the order in which the plurality of operations is executed. Therefore, when each example embodiment is implemented, the order of the plurality of operations can be changed within a range that does not interfere with the content.

Further, it is noted that the inventor's intent is to retain all equivalents of the claimed disclosure even if the claims are amended during prosecution.

Some or all of the above example embodiments may be described as the following Supplementary Notes, but are not limited to the following.

### Supplementary Note 1

A determination device including
an acquisition means for acquiring electronic receipt information about a patient,
a determination means for determining necessity of a detailed description of a symptom of the patient based on the electronic receipt information about the patient using a learned model that has learned electronic receipt information excluding a detailed description of a symptom in a past and a condition requiring a detailed description of a symptom, and
an output means for outputting a determination result.

### Supplementary Note 2

The determination device according to Supplementary Note 1, wherein
the acquisition means acquires electronic receipt information about a plurality of patients for each of whom a medical act has been performed at a predetermined time,
the determination means determines necessity of the detailed description of the symptom of each patient, and
the output means outputs a list of patients for each of whom the detailed description of the symptom is required.

### Supplementary Note 3

The determination device according to Supplementary Note 1 or 2, wherein the output means outputs a basis of electronic receipt information determined to require the detailed description of the symptom.

### Supplementary Note 4

The determination device according to Supplementary Note 2, wherein the output means outputs a list of patients for each of whom the detailed description of the symptom is required for a diagnosis and treatment department name or a doctor in charge of each patient.

### Supplementary Note 5

The determination device according to Supplementary Note 1 or 2, wherein the learned model is a model that has learned a relationship between electronic receipt information including at least any one of a sick name, content of a medical act, and the score of a claim for a medical fee, and necessity of the detailed description of the symptom.

### Supplementary Note 6

The determination device according to Supplementary Note 1 or 2, wherein the acquisition means acquires electronic receipt information about a patient related to a logged-in user

### Supplementary Note 7

The determination device according to Supplementary Note 1 or 2, further including a reception means for receiving a correction of the output determination result.

### Supplementary Note 8

The determination device according to Supplementary Note 7, further including a learning means for causing the learned model to relearn electronic receipt information that has received the correction and necessity of the detailed description of the symptom as learning data.

### Supplementary Note 9

A determination method executed by a computer, the method including
acquiring electronic receipt information about a patient,
determining necessity of a detailed description of a symptom of the patient based on the electronic receipt information about the patient using a learned model that has learned electronic receipt information excluding a detailed description of a symptom in a past and a condition requiring a detailed description of a symptom, and
outputting a determination result.

### Supplementary Note 10

A program for causing a computer to execute
acquiring electronic receipt information about a patient,
determining necessity of a detailed description of a symptom of the patient based on the electronic receipt information about the patient using a learned model that has learned electronic receipt information excluding a detailed description of a symptom in a past and a condition requiring a detailed description of a symptom, and
outputting a determination result.

Some or all of the configurations described in Supplementary Notes 2 to 8 dependent on the above-described Supplementary Note 1 can also be dependent on Supplementary Notes 9 and 10 by the dependency relationship similar to that of Supplementary Notes 2 to 8. Furthermore, some or all of the configurations described as the Supplementary Notes can be similarly dependent on not only the Supplementary Notes 1, 9, and 10, but also various pieces of hardware and software, and various recording devices or systems for recording software without departing from the above-described example embodiments.

### REFERENCE SIGNS LIST

100, 110 determination device
101, 111 acquisition unit
102, 112 determination unit
103, 113 output unit
114 reception unit
115 learning unit
500 computer device
501 CPU
502 ROM
503 RAM
504 program
505 storage device
506 recording medium
507 drive device
508 communication interface
511 input/output interface
512 bus

1. A determination device comprising:
at least one memory configured to store instructions; and
at least one processor configured to execute the instructions to:
acquire electronic receipt information about a patient;
determine necessity of a detailed description of a symptom of the patient based on the electronic receipt information about the patient using a learned model that has learned electronic receipt information excluding a detailed description of a symptom in a past and a condition requiring a detailed description of a symptom; and
output a determination result.

2. The determination device according to claim 1, wherein the at least one processor is further configured to execute the instructions to:
acquire electronic receipt information about a plurality of patients for each of whom a medical act has been performed at a predetermined time;
determine necessity of the detailed description of the symptom of each patient; and
output a list of patients for each of whom the detailed description of the symptom is required.

3. The determination device according to claim 1, wherein the at least one processor is further configured to execute the instructions to:
output a basis of electronic receipt information determined to require the detailed description of the symptom.

4. The determination device according to claim 2, wherein the at least one processor is further configured to execute the instructions to:
output a basis of electronic receipt information determined to require the detailed description of the symptom.

5. The determination device according to claim 2, wherein the at least one processor is further configured to execute the instructions to:
output a list of patients for each of whom the detailed description of the symptom is required for a diagnosis and treatment department name or a doctor in charge of each patient.

6. The determination device according to claim 1, wherein
the learned model is a model that has learned a relationship between electronic receipt information including at least any one of a sick name, content of a medical act, and a score of a claim for a medical fee, and necessity of the detailed description of the symptom.

7. The determination device according to claim 1, wherein the at least one processor is further configured to execute the instructions to:
acquire electronic receipt information about a patient related to a logged-in user.

8. The determination device according to claim 1, wherein the at least one processor is further configured to execute the instructions to:
receive a correction of the output determination result.

9. The determination device according to claim 8, wherein the at least one processor is further configured to execute the instructions to:
cause the learned model to relearn electronic receipt information that has received the correction and necessity of the detailed description of the symptom as learning data.

10. A determination method comprising:
acquiring electronic receipt information about a patient;
determining necessity of a detailed description of a symptom of the patient based on the electronic receipt information about the patient using a learned model that has learned electronic receipt information excluding a detailed description of a symptom in a past and a condition requiring the detailed description of the symptom; and
outputting a determination result.

11. The determination method according to claim 10, further comprising:
acquiring electronic receipt information about a plurality of patients for each of whom a medical act has been performed at a predetermined time;
determining necessity of the detailed description of the symptom of each patient; and
outputting a list of patients for each of whom the detailed description of the symptom is required.

12. The determination method according to claim 10, further comprising:
outputting a basis of electronic receipt information determined to require the detailed description of the symptom.

13. The determination method according to claim 11, further comprising:
outputting a basis of electronic receipt information determined to require the detailed description of the symptom.

14. The determination method according to claim 11, further comprising:
outputting a list of patients for each of whom the detailed description of the symptom is required for a diagnosis and treatment department name or a doctor in charge of each patient.

15. A non-transitory computer-readable recording medium that records a program for causing a computer to execute:
acquiring electronic receipt information about a patient;
determining necessity of a detailed description of a symptom of the patient based on the electronic receipt information about the patient using a learned model that has learned electronic receipt information excluding a detailed description of a symptom in a past and a condition requiring a detailed description of a symptom; and
outputting a determination result.

16. The recording medium, according to claim 15, that records the program for causing the computer to further execute:
acquiring electronic receipt information about a plurality of patients for each of whom a medical act has been performed at a predetermined time;
determining necessity of the detailed description of the symptom of each patient; and
outputting a list of patients for each of whom the detailed description of the symptom is required.

17. The recording medium, according to claim 15, that records the program for causing the computer to further execute:
outputting a basis of electronic receipt information determined to require the detailed description of the symptom.

18. The recording medium, according to claim 16, that records the program for causing the computer to further execute:
outputting a basis of electronic receipt information determined to require the detailed description of the symptom.

19. The recording medium, according to claim 16, that records the program for causing the computer to further execute:
outputting a list of patients for each of whom the detailed description of the symptom is required for a diagnosis and treatment department name or a doctor in charge of each patient.
